(19) United States
(12) Patent Application Publication
(10) Pub. No.: US 2008/0317662 A1
(43) Pub. Date: Dec. 25, 2008

(54) Molten Salts, Method of Their Production and Process for Generating Hydrogen Peroxide

(75) Inventor: Andrew P. Doherty, Belfast (GB)

Correspondence Address: PEPPER HAMILTON LLP, ONE MELLON CENTER, 50TH FLOOR, 500 GRANT STREET, PITTSBURGH, PA, 15219, US

(73) Assignee: THE QUEENS UNIVERSITY OF BELFAST, Belfast (GB)

(21) Appl. No.: 11/571,462

(22) PCT Filed: Jun. 30, 2005
(86) PCT No.: PCT/GB2005/002565

§ 371 Date: Aug. 27, 2008

(30) Foreign Application Priority Data
Jun. 30, 2004 (GB) 0414597.5

(51) Int. Cl. C01B 15/023; C01B 15/022; C07D 233/54; C07D 207/00; C07C 50/04; C07C 50/18

(52) U.S. Cl. 423/588; 548/334.5; 548/530; 552/209; 552/294

(57) ABSTRACT

A molten salt and process for preparing a molten salt or hydrogen peroxide uses ionic hydroquinones or hydroquinone derivatives as O₂ reduction catalysts.

### CROSS-REFERENCE TO RELATED APPLICATIONS

This patent application claims priority to Great Britain Patent Application Number 0414597.5 with a Filing Date of Jun. 30, 2004. The application was also filed as International Patent Application PCT/GB2005/002565 with an International Filing Date of Jun. 30, 2005, with subsequent publication as International Publication Number WO 2006/003395 on January 12, 2006. The disclosures of each of the aforementioned patent documents are incorporated herein by reference in its entirety.

### STATEMENT REGARDING FEDERALLY SPONSORED RESEARCH

Not applicable.

### NAMES OF PARTIES TO A JOINT RESEARCH AGREEMENT

Not applicable.

### SEQUENCE LISTING

Not applicable.

### BACKGROUND

Hydrogen peroxide (H₂O₂) is one of the world's most important bulk inorganic chemicals with current global production in excess of 2 million tonnes per annum. The chemistry associated with the anthraquinone autooxidation process (AOP) by which H₂O₂ is predominantly manufactured is shown in scheme 1.

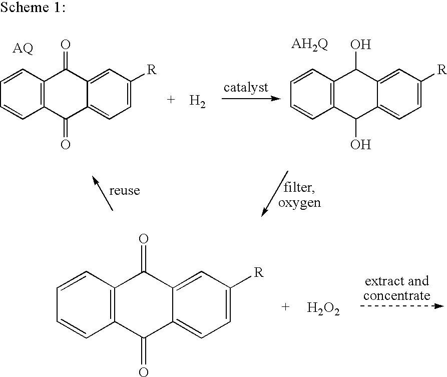

The process involves dissolving a substituted anthraquinone (AQ-R, R=hydrocarbon group) in a water-immiscible solvent (or solvent mixture) such as tetramethylbenzene. R-substitution of the anthraquinone ensures maximum solubility ill the organic phase while maintaining minimum solubility in the extraction aqueous phase. The anthraquinone is subsequently catalytically reduced to the anthrahydroquinone (AlH₂Q) using H_{2(g)} under pressure in the presence of a hydrogenation catalyst such as supported Pd or Pt. The supported catalyst is then removed by filtration. Passing O_{2(g)} (usually in air or as pure O₂) through the resultant solution results in the highly selective 2 electron/2 proton reduction (otherwise known as hydrogenation) of O₂ to H₂O₂ accompanied by the 2 electron/2 proton oxidation (otherwise known as dehydrogenation) of AH₂Q back to AQ. The hydrogen peroxide is then recovered from the organic solvent media phase by extraction into an immiscible water phase. Addition of water is generally concomitant with the addition of oxygen. Alter extraction, the AQ solution is reused within the process while the aqueous H₂O₂ is concentrated via H₂O evaporation. Typical production facilities have capacities of 40,000 to 60,000 tonnes per annum, such facilities are usually located in regions of high peroxide consumption.

The AOP approach is used because of its selectivity, and therefore, its high atom efficiency and also because of the relative ease with which pure aqueous solutions of peroxide can be obtained. Notwithstanding, considerable effort exists to find alternative routes to peroxide.

One alternative route is based on the direct heterogeneous catalytic reaction of hydrogen and oxygen in aqueous solution. In such a process, the reaction medium is an acidic solution containing halide ions. Inevitably, the use of such a corrosive liquid has a detrimental effect both on the catalyst stability and the reactor, and results in a complex aqueous mixture from which the H₂O₂ must be isolated and the catalyst recovered. One approach to addressing these problems has been to incorporate both the halide ions and acid functions into the solid catalyst. The halide, which promotes the Pt-group metal catalyst, is provided as an insoluble organo-silane precursor; and the acid function is provided by using acidic or super acid solids as the catalyst support.

A homogeneous alternative to the above route is disclosed in U.S. Pat. No. 4,336,240, which is incorporated by reference herein in its entirety, wherein the reaction medium comprises an immiscible (biphasic) mixture of water and an organic fluorocarbon solvent in which an organometallic Pd-catalyst is dissolved. On formation, the hydrogen peroxide is dissolved in the aqueous phase, preventing further catalytic reaction (to H₂O). A similar approach is disclosed in U.S. Pat. No. 4,347,232 which is incorporated by reference herein in its entirety, except that in this case the catalyst (a dibenzylidene acetone complex of palladium) is dissolved in chlorobenzene. This type of homogeneous/bi-phasic reaction has the drawback of producing H₂O₂ in low concentrations.

In order for direct routes to compete with the AOP approach, they should advantageously have comparable H₂O₂-formation efficiency and preferably lower capital, separation and catalyst-recycling costs. However, existing processes (both heterogeneous and homogeneous) show a recurrence of one or more of the following limitations: low rate of H₂O₂ formation; finite solubility of (heterogeneous) catalyst in the reaction medium; difficult separation of H₂O₂ from reaction medium; poor performance of homogeneous catalyst; (frequently reaction) can only be carried out in batch mode; organic solvents must be used; and high pressure is required (leading to widening of flammability window and high capital cost of compression).

Accordingly there remains a need to develop a H₂O₂ generation process which addresses these limitations.

Furthermore, for a variety of reasons. including the explosive nature of H₂O₂ and its frequent use in remote locations, there is considerable interest in developing technology for on-site on-demand peroxide generation so as to avoid transport/storage hazards and associated costs.

The electrolytic production of hydrogen peroxide has been known since the nineteenth century. For many years the primary method of manufacturing hydrogen peroxide was by electrolysis using a route where persulfate is formed at an anode and then hydrolysed (Kirk-Othmer Encyclopaedia of Chemical Technologies, 3rd Edition, Volume 13, (1981)). An approach based on the direct electrochemical reduction of O2 to H₂O₂ at gas diffusion electrodes has been developed. Typically, reduction occurs at old gas diffusion electrodes in alkaline electrolytes with H₂O oxidation occurring at a Pt anode. In this arrangement, O₂ generated at the anode from H₂O oxidation, as well as atmospheric O₂, is fed to the cathode to be reduced to peroxide. This approach generates an alkaline solution of hydrogen peroxide that can be used directly in many applications e.g. pulping/bleaching.

An alternative indirect electrolytic strategy, that combines the heterogeneous nature of electrochemistry with the selectivity/efficiency of the hydroquinone approach, has been demonstrated (see for example Hoang et al, J. Electrochem Soc. 132 (1985) pp. 2129-2133; and DeGrand et al, J. Electroanalytical Chem. 169(1984) pp 259-268, ibid 117(1981) pp. 267-281). In this approach, polymeric materials possessing pendant anthraquinone functional groups axe attached to electrode surfaces. In the presence of a proton (H⁺) source, the anthraquinone can be electrolytically converted into anthrahydroquinone by direct electron transfer from the electrode accompanied by protonation from the electrolyte. There has also been disclosure of al indirect electrochemical means for generating hydrogen peroxide where an electrochemical cell is used to reduce quinone species anchored to high surface area support particles suspended in electrolyte solution (see for example U.S. Pat. No. 4,533,443, U.S. Pat. No. 4,533,443, and U.S. Pat. No. 4,572,774, the disclosures of which are each incorporated herein in their entirety). The suspended particles are removed from the cell and reacted with oxygen to produce hydrogen peroxide. The oxidized anchored quinone is subsequently returned to the electrolytic cell for re-reduction.

Although the concept of small-scale on-site electrolytic generation of peroxide is attractive., such technology is unable to supply the volume demands for the majority of peroxide users. For this reason, this approach is viewed as only potentially useful for particular niche markets rather than an alternative to the large-scale production and therefore, the AOP process continues to be the main global source of bulk peroxide.

### SUMMARY

While the AOP is the predominant manufacturing technology for peroxide generation it is widely considered to be unsustainable because it requires vast quantities of volatile toxic solvents, produces associated toxic emissions and is notoriously hazardous (explosive risk of H₂O₂ combined with volatile organic solvents). In order to render it less hazardous, total elimination of organic solvents from the process would be desirable. It is an object of the present invention to provide a process for generating H₂O₂ which represents an alternative to the processes described above.

It is therefore an object of the present invention to provide an alternative to the solvent based and electolytic processes for the preparation of hydrogen peroxide which address limitations of the prior art processes discussed above.

It is a further object of the invention to provide a class of molten salts which may be used as catalysts, and in particular as homogeneous catalysts of reactions such as the redox production of hydrogen peroxide.

### BRIEF DESCRIPTION OF THE DRAWINGS

FIG. 1 shows the infrared (IR) spectra of butylmethylpyrrolidinium hydroquinonesulfonate.

FIG. 2 shows the IR spectra of butylmethylimidazolium hydroquinonesulfonate.

FIG. 3 shows the IR spectra of butylmethylpyrrolidinium anthraquinone-2-sulfonate.

FIG. 4 shows the IR spectra of butylmethylimidazolium anthraquinone-2-sulfonate.

FIG. 5 shows the IR spectra of tetraphenylphosphonium hydroquinone sulfonate.

FIG. 6 shows the IR spectra of butylmethylpyrrolidinium anthraquinone-2-carboxylate.

FIG. 7 shows the IR spectra of N-butyl-N-methyl piperidinium hydroquinone sulfonate.

FIG. 8 shows the IR spectra of N-octyl-N-methyl piperidinium hydroquinone sulfonate.

FIG. 10 shows the IR spectra of tetradecyltrihexylphosphonium hydroquinone sulfonate.

FIG. 11 is a current-voltage profile for butylmethylimidazolium anthraquinone-2-carboxylate.

FIG. 12 is a series of current-voltage profiles for 1.0×10⁻³ mol dm⁻³ butylmethylimidazolium anthraquinone-2-carboxylate in acetonitrile with 1.0×10⁻³ mol dm⁻³ tetrabutylammonium tetrafluoroborate and 0.1 mol dm⁻³ benzoic acid.

FIG. 13 is a series of cyclic voltammograms for the detection of hydrogen peroxide.

### DETAILED DESCRIPTION

Accordingly, the present invention provides, in general terms, a class of molten salts, useful as catalysts, a process for the production of said molten salts and a process for the preparation of hydrogen peroxide which uses ionic hydroquinones (or hydroquinone derivatives) as homogeneous O₂ reduction catalysts preferably in the absence of molecular solvents.

According to a first aspect of the present invention there is provided a molten salt (Cat⁺An⁻) that includes a quinone or quinone derivative as anion or cation. The quinone or quinone derivative may have the structure of Formula I, II or III.

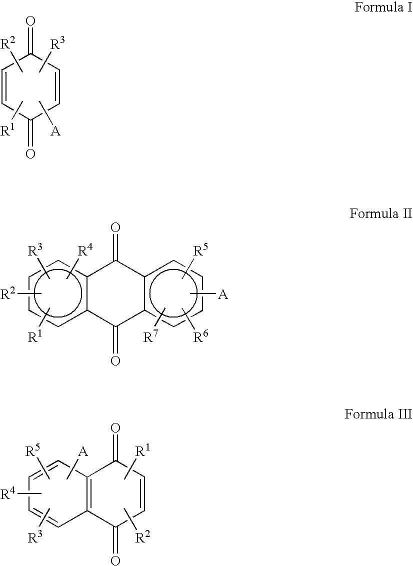

wherein:

one or more of any ring atom of any one of Formulae I-III may be a heteroatom, such as N, S, O or P, that may suitably be quaternised to from a cationic species;

the position of the carbonyl species of any one of Formulae I to III (C═O) may be anywhere on any of the rings;

R¹ to R⁷ may independently be A; hydrogen; C₁₋₁₀ linear, branched chain or cyclic alkyl groups; aryl; heterocycles; CN; OH; or NO₂ wherein said alkyl and aryl substituents may themselves be substituted or unsubstituted;

if the quinone or quinone derivative is anionic A represents SO₃⁻ or COO⁻, and if the quinone or quinone derivative is cationic either: A and optionally one or more of R¹-R⁷ independently represent imidazolium, piperidinium, pyridinium, phosphonium, pyrazinium, quaternary amine, ammonium species or derivatives thereof or one or more of the ring atoms is a quaternised heteroatom and each quaternised heteroatom may independently represent an imidazolium, piperidinium, pyridinium, phosphonium, pyrazinium, quaternary amine, imonium species or derivatives thereof and A represents hydrogen: a C₁₋₁₀ linear, branched chain or cyclic alkyl group, an aryl group; a heterocycle group; CN; OH or NO₂ where the alkyl and aryl substituents may themselves be substituted or unsubstituted.

The term aryl includes for example phenyl, polyphenyl, benzyl and similar moieties.

The term quinone derivative includes quinone, naphthoquinone, hydroquinone and anthroquinone derivatives.

In one embodiment the molten salt consists of cations and anions only.

For the purposes of describing the invention anions of Formulae I, II and III are referred to collectively as An⁻.

If the quinone or quinone derivative is anionic it typically has a hydroquinone structure:

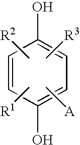

In one embodiment the quinone derivative has the following structure

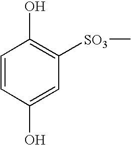

Alternatively the anionic quinone or quinone derivative has the structure:

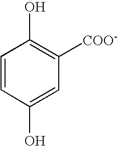

If the quinone or quinone derivative is anionic the cation (Cat⁺) of the molten salt is suitably an aliphatic or aromatic hydrocarbon species typically possessing a hetero-atom, such as N, S, P and O. The aliphatic or aromatic hydrocarbon species may be substituted or unsubstituted, typically with one or more of any substituted or unsubstituted alkane, alkene, alkyne or aromatic hydrocarbon or any halogen group such as a fluorocarbon group. The cation may include one or more amine, aide, nitrile, halogen, ether, alcohol, thiol, acid, ester, aldehyde, ketone or phosphine group. Suitably the cation comprises a branched alkyl chain such as a fluorinated branched alkyl chain. In one embodiment the cation is tetraalkylphosphonium.

Alternatively the cation may be selected from the group consisting of imidazolium, piperidinium, pyridinium, phosphonium, pyrrolidinium, pyrazinium, quaternary amine, ammonium species and derivatives thereof, Suitably the cation is selected from the group consisting of imidazolium, piperidinium, phosphonium quaternary amine and ammonium species.

When Cat⁺ is an imidazolium cation it is preferably a cation of Formula IV:

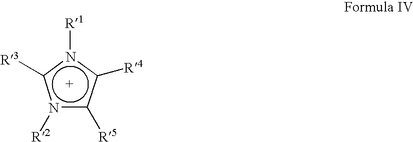

In one embodiment the cation is:

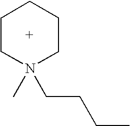

When Cat⁻ is a piperidinium cation it is preferably a cation of Formula V:

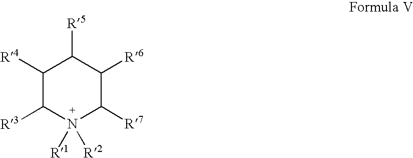

In one embodiment the cation is,

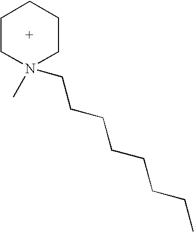

Alternatively the cation is:

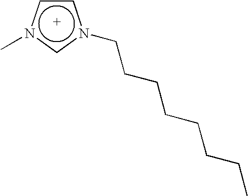

When Cat⁺ is a pyridinium cation it is preferably a cation of Formula VI:

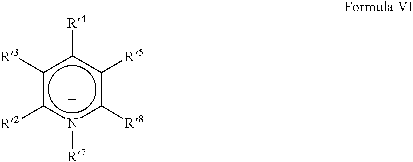

When Cat⁻ is a phosphonium cation it is preferably an cation of Formula VII:

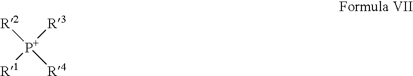

In one embodiment the cation is tetradecyltrihexylphosphonium and has the structure:

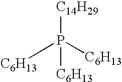

Where they appear in Formulae IV to VII R′¹ to R′⁷ may independently be hydrogen, a substituted or unsubstituted C₁₋₁₀ linear or branched alkyl chain a substituted or unsubstituted cyclic alkyl group, an aryl group, CN, OH, NO₂, SO₃ or COO.

When Cat⁺ is a quaternary amine it is preferably of the form NR₄⁺ where each R is independently a substituted or unsubstituted C₁₋₂₀ linear or branched alkyl chain or a substituted or unsubstituted cyclic alkyl group. Suitably the alkyl groups may be substituted with one or more alkane, alkyne or aromatic hydrocarbon or any halogen group such as a fluorocarbon group.

If the quinone or quinone derivative is cationic it typically has the structure:

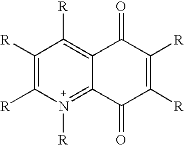

If the quinone or quinone derivative is cationic the anion of the molten salt is any suitable anionic species such as PF₆, tetrafluoroborate, bistriflimide, triflate, nitrate, a phosphate such as hexafluorophosphate, carboxylic acid, dicyanamide or thiocyanate.

In one embodiment the molten salt has a melting point of less than 100° C. preferably less than 0° C. Suitably the molten salt consist entirely of anions and cations. The preferred molten salt is preferably as hydrophobic as possible.

In one embodiment the molten salt is N-butyl-N-methyl piperidinium hydroquinone sulfonate. Alternatively the molten salt may be N-octyl-N-methyl piperidinium hydroquinone sulfonate or 1-octyl4-methyl imidazolium hydroquinone sulfonate. In a further embodiment the molten salt may be tetradecyltrihexylphosphonium hydroquinone sulfonate, butylmethylimidazolium hydroquinonesulfonate, butylmethylpyrrolidinium hydroquinonesulfonate or butylmethyl imidazolium anthraquinone-2-carboxylate.

In one embodiment the molten salt is an ionic liquid.

According to a further aspect of the present invention there is provided a mixture of two or more of the abovementioned molten salts, or combination of ions thereof.

The present invention further provides a method of preparing a molten salt (Cat⁺An⁻) as described above including the steps of:

(a) dissolving a first salt nCat⁺Xⁿ⁻, where X=Cl⁻, Br⁻ or I⁻ in which case n=1, or X=SO₄²⁻ in which case n=2, in an organic solvent.

(b) dissolving a second salt xM⁺An^{x-}, where M=K⁺, Na⁺, Li⁺ or Ag⁺ and x=1 to 8, in an organic solvent;

(c) precipitating the inorganic salt (nMXⁿ⁻) by mixing the solutions formed according to steps (a) and (b)l and

(d) removing the organic solvent to recover the molten salt (Cat⁺An⁻).

Optionally the inorganic salt (nMXⁿ⁻) is removed from the solution through filtration.

Preferably the solvent used in either or both of steps (a) and (b) is selected from the group consisting of acetonitrile, acetone, dimethylformamide, tetrahydrofuran, dimethylsulfoxide and mixtures thereof.

The molten salt thus produced may be purified by redissolving in an organic solvent, such as those listed above, filtration and removal of the solvent.

According to a further aspect of the present invention there is provided an alternative method of preparing a molten salt (Cat⁺An⁻) as described above including the step of:

(A) heating, in the solid state, a mixture of a carboxylic or sulfonic acid (bH⁺An^{b-}) where b=1 to 8 and a salt (nCat⁺Xⁿ⁻) (as defined above) liberating nH⁺Xⁿ⁻; and

(B) recovering the molten salt (Cat⁺An⁻)

Suitably a solvent is added to the mixture, dissolving the molten salt (Cat⁺An⁻). The solvent is then suitably removed from the molten salt under vacuum. The solvent may be organic. Preferably the solvent is acetonitrile, acetone, dimethylformamide, tetrahydrofuran, dimethylsulfoxide or mixtures thereof,

The present invention provides a catalyst comprising the molten salt (Cat⁺An⁻) as described above suitable, for example, in the production of hydrogen peroxide.

The present invention also provides a process for the production of hydrogen peroxide comprising the step of:

oxidising a molten salt comprising a hydroquinone or hydroquinone derivative as anion (An⁻) or cation (Cat⁺) to form the corresponding quinone or quinone derivative and produce hydrogen peroxide.

In one embodiment the process comprises the step of reducing a molten salt comprising a quinone or quinone derivative as anion (An⁻) or cation (Cat⁺) to produce the hydroquinone or hydroquinone derivative.

Preferably the process is carried out substantially in the absence of any molecular solvent.

The reduction step may be effected by any suitable means such as, for example, catalytic hydrogenation or electrolysis. Suitably the reduction step involves contacting the molten salt with H₂ suitably with a supported or unsupported metal hydrogenation catalyst such as palladium, platinum and nickel under a pressure of up to 60 bar.

In one embodiment of the invention, the process may optionally comprise the step of adding an ionic liquid to the molten salt comprising a hydroquinone or hydroquinone derivative. Suitably the ionic liquid comprises imidazolium, pyridinium, piperidinium, phosphonium or quaternary ammonium salts of triflate, bistriflimide, nitrate hexafluorophosphate and tetrafluoroborate.

In one embodiment the reduction step takes place in the presence of one or more organic solvents such as alcohols, alkanes, nitrites etc. The presence of organic solvents may enhance the reduction step or may facilitate further processing.

The oxidation step may be effected by any suitable means such as contacting the hydroquinone or hydroquinone derivative with oxygen, or with air and water. Suitably the hydroquinone or hydroquinone derivative is contacted with air and water to produce biphasic products wherein H₂O₂ is in the water phase.

Preferably the molten salt is as described above.

The invention also provides for the use of the molten salt as described above in a process for the preparation of hydrogen peroxide using a homogeneous O₂ reduction catalyst which is itself in the form of a molten salt.

Various molten salts or combinations of salts composed entirely of cations and anions are known which may he useful as alternatives to conventional reaction media. The process of the invention disclosed herein employs hydroquinones or hydroquinone derivatives as homogenous O₂ reduction catalysts, preferably in the absence of molecular solvents. This is effected by synthesising the molten salts described above. Any combination of the aforementioned anions and cations may be used in the synthesis of a mixed molten salt suitable for use in the process of the invention (i.e. the molten salt used in the invention may comprise more than one anion and/or cation).

In effect the present invention provides for an immobilised hydroquinone redox catalyst in liquid molten salt form in a medium which may be substantially free of molecular solvents. This contrasts with the conventional auto-oxidation process where the catalytic hydroquinone species is dissolved in an organic solvent or solvent mixture. Therefore, the catalytic process of the invention is capable of generating peroxide substantially in the absence of organic solvent. Furthermore, since the hydroquinone/quinone catalyst comprises up to 50 mole % of the molten salt, extremely high catalyst loading can be obtained. Further advantages of the process for the production of hydrogen peroxide of the invention include:

the redox catalyst is in the form of a processable liquid;

the redox catalyst is the highly selective/efficient quinone moiety;

the process may be carried out in the absence of any, or any substantial amount, of conventional solvents;

non-volatile, non-flammable, non-explosive catalytic medium;

high catalyst loading;

amenable to both small-scale electrolytic generation and catalytic H₂ generation of peroxide;

the process of the present may have through-puts significantly exceeding the AOP approach; and

the process may have greater space-time yields than the AOP reaction.

### EXAMPLES

### Example 1

### Synthesis of Quinone-Containing Molten Salts

Synthesis of the aforementioned catalytic molten salts may be effected as follows:

1) ion metathesis reaction of a halide salt (X⁻) of the aforementioned cations (or combination thereof with a metal salt (Mⁿ⁺) of the carboxylate and/or sulfonate substituted quinones. Typically, this may be carried out in any suitable organic solvent (or solvent mixture) such as for example dimethylformamide (DMF), acetone, acetonitrile, ethanol or methanol (and mixtures thereof). In such solvents the insoluble inorganic salt Mⁿ⁺nX⁻ precipitates and may be removed by filtration. The solvent may be removed from the filtrate by evaporation and the resultant product (molten salt) recovered. The product may then be purified by repeated dissolution in organic solvent with any residual insoluble Mⁿ⁺nX⁻ removed by filtration.

The molten salts (1.1-2.2) listed below were made by preparing and mixing separate solutions of the anion and cation in volumes appropriate to give stoichiometric quantities of each. The concentration of anion and cation solutions used were typically in the order of 10% wt/vol in the solvent in question. All quinone anion salts were dissolved in DMF, while acetonitrile was used to dissolve all imidazolium and pyrrolidinium cation salts. Tetraphenylphosphonium salts were dissolved in DMF, although ethanol was found to be a useful alternative for phosphonium salts. The reactions were carried out at room temperature under stirring conditions for 24 hours. The molten salt product was recovered as outlined above. Yields were quantitative and determined to be approximately 100% in each case.

2) Reaction of the carboxylic or sulfonic acid derivatives of the quinone or hydroquinone with the halide (X⁻) salt of the aforementioned cations. This reaction may be carried out in the solid-state with gentle heating to initiate the reaction which results in evolution which may be removed by vacuum.

The following salts were synthesised according to the above procedure (melting points shown in brackets):

| | | |
|---|---|---|
| 1.1 | [Bmpyr]⁺[HQS]⁻ | (105-107° C.); |
| 1.2 | [Bmim]⁺[HQS]⁻ | (<−20° C.); |
| 1.3 | [Bmpyr]⁺[AQS]⁻ | (108-115° C.); |
| 1.4 | [Bmim]⁺[AQS]⁻ | (153° C.); |
| 1.5 | [Bmim]⁺[AQCOO]⁻ | (97° C.); |
| 1.6 | [TPP]⁺[HQS]⁻ | (240° C.); |
| 1.7 | [BTFAP]⁺[AQS]⁻; | |
| 1.8 | [Bmpyr]⁺[AQCOO]⁻; | |

| | |
|---|---|
| 1.9 | N-butyl-N-methyl piperidinium hydroquinone sulfonate; |
| 2.0 | N-octyl-N-methyl piperidinium hydroquinone sulfonate; |
| 2.1 | 1-octyl-4-methyl imidazolium hydroquinone sulfonate; and |
| 2.2 | tetradecyltrihexylphosphonium hydroquinone sulfonate; |

where
[Bmim]⁺ = butylmethylimidazolium,
[Bmpyr]⁺ = butylmethylpyrrolidinium,
[TPP]⁺ = tetraphenylphosphonium,
[BTFAP]⁺ = 2-[N,N-bis(trifluoromethanesulfonyl)amino pyridinium,
[HQS]⁻ = hydroquinonesulfonate,
[AQS]⁻ = anthraquinone-2-sulfonate and
[AQCOO]⁻ = anthraquinone-2-carboxylate.

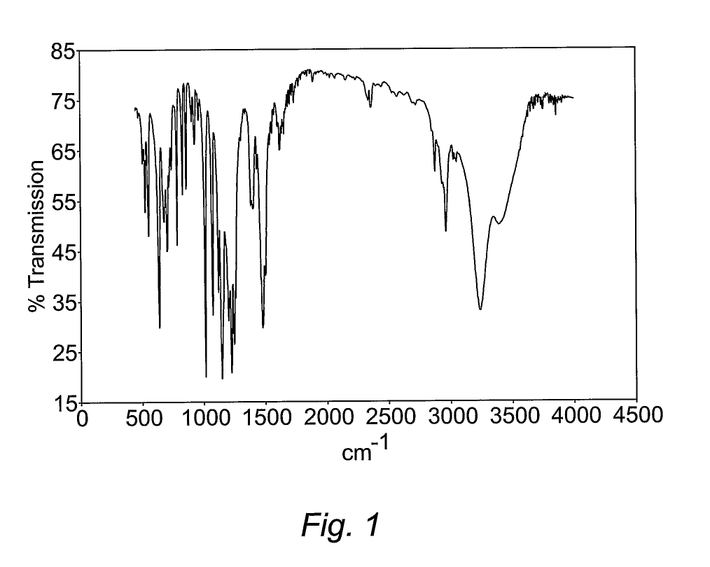

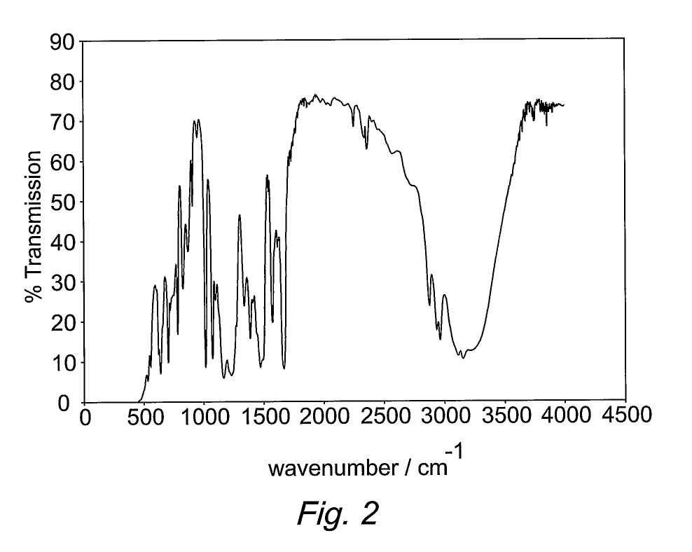

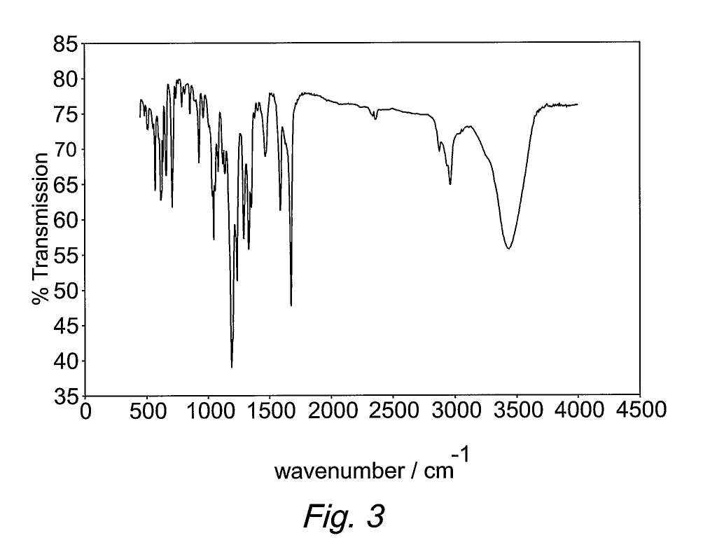

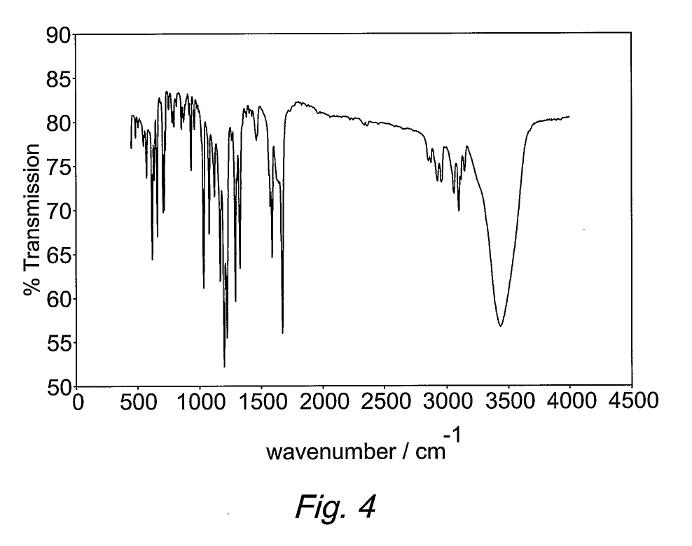

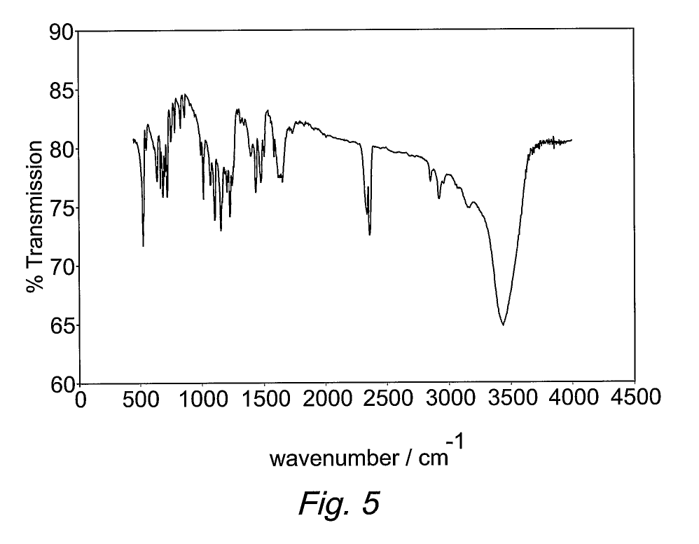

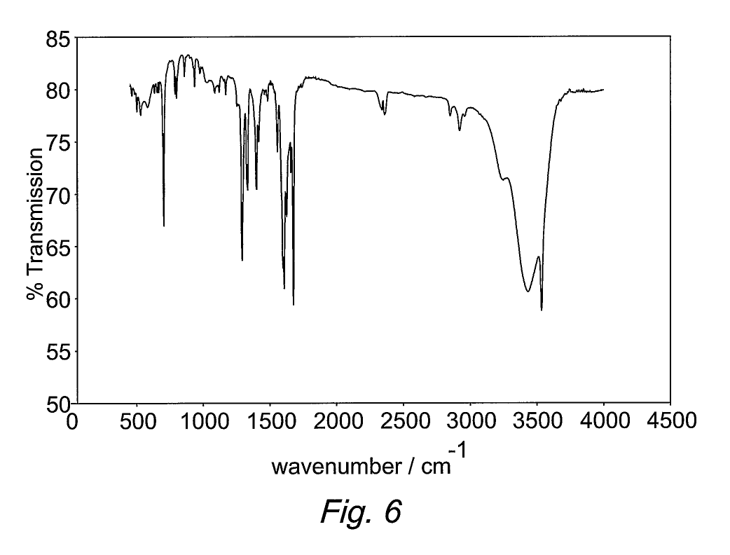

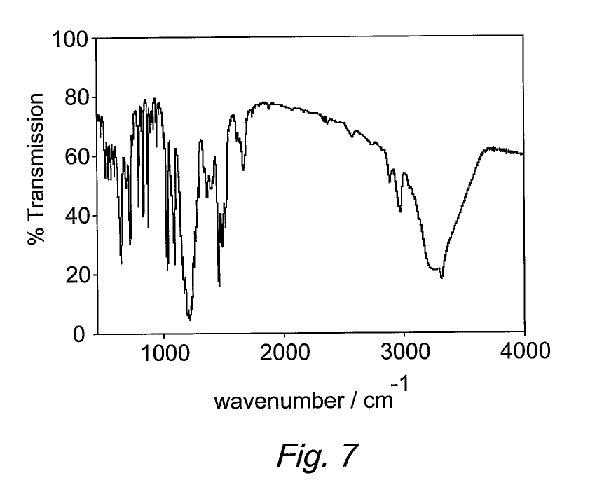

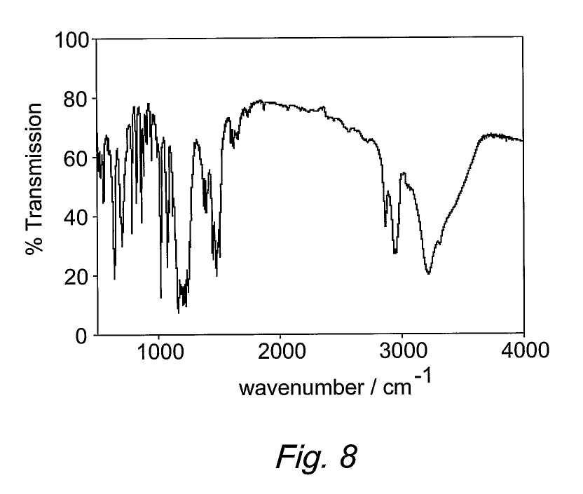

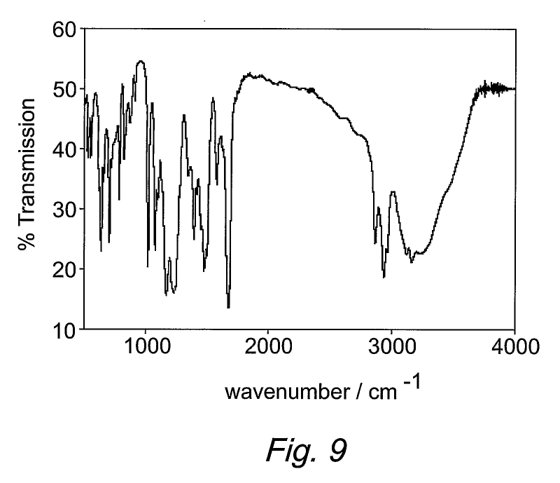

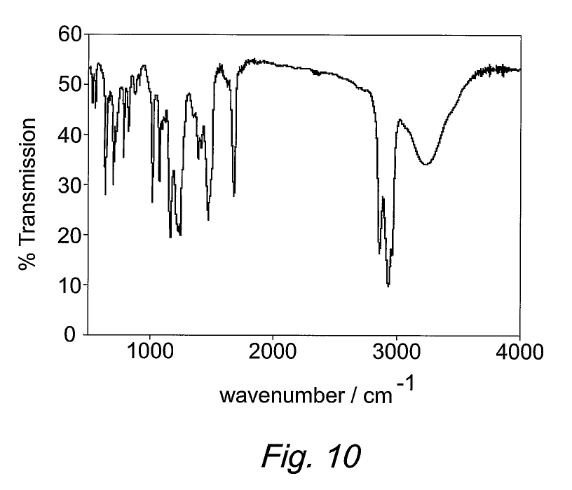

FIGS. 1 to 10 show infrared spectra for compounds 1.1, 1.2, 1.3, 1.4, 1.6 ad 1.8 to 2.2 respectively. IR spectra were recorded using a Perkin-Elmer ‘Spectrum RX/FT-IR’ spectrometer with a resolution of4 cm⁻¹. Samples which were solid at room temperature were prepared as KBr disks, while samples which were liquid at room temperature were prepared as pure liquid films between NaCl plates.

### Example 2

### Assessment of Catalytic Activity of Molten Salts for O₂ Reduction

Activation of the quinone (or quinone derivative) species to the catalytically active hydroquinone (or anthrahydroquinone) may be effected by catalytic H_{2(g)} reduction or by reductive electrolysis at an electrode in the presence of a proton source. At catalytic electrodes such as Pd or Pt, the reaction is identical to the H_{2(g) approach.}

### Example 2.1

Electrolytic reduction of the molten salt [Bmim^{═}] [AQ-COO⁻] (where [Bmim⁺] is 1-butyl-3-methylimidazolium and [AQ-COO⁻] is 9,10-anthraquinone-2-carboxylate) in the pure state and dissolved in an organic solvent (acetonitrile with tetrabutylammonium borate electrolyte):

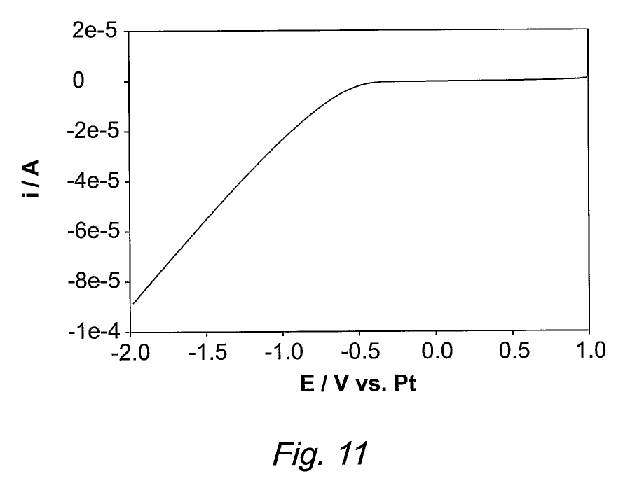

FIG. 11 shows the current (i) versus electrode potential for the pure molten salt. It can be seen that the current (negative cathodic current) begins to increase monotonically from −0,5 V. The cathodic current response is due to the reduction of the anthraquinone species which clearly indicates the retention of anthraquinone/hydroquinone electrochemical activity in the molten salt. In order to assess the electrochemical activity of the [Bmim⁺] [AQ-COO⁻] in the absence and presence of O₂, the salt was dissolved in acetonitrile to give a 1.0×10⁻² mol dm⁻³ solution of [Bmim⁺] [AQ-COO⁻] along with 1.0×10⁻² mol dm⁻³ tetrabutylammonium tetrafluoroborate electrolyte and 0.1 mol dm⁻³ benzoic acid acting as the proton source.

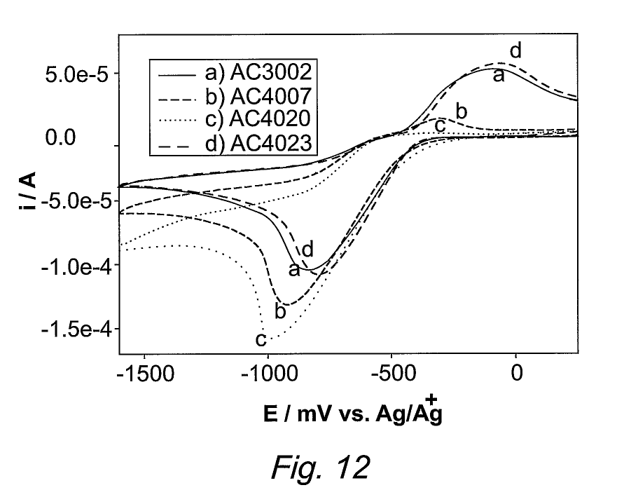

FIG. 12a shows the cyclic voltammogram for the [Bmim⁺] [AQ-COO⁻] under O₂-free conditions where a broad reduction process occurs at −0.85 V vs. Ag/Ag⁺ due to the two electron/two proton reduction of the anthraquinone to the anthrahydroquinone. On the reverse voltage sweep, a reoxidation process is observed which is due to the oxidation of the anthrahydroquinone back to the anthraquinone.

FIGS. 12b and 12c show voltamnmograms recorded as O₂ is emitted to the electrochemical cell. Time open to the atmosphere is the variable, curve a) is a t time=0, curve b) is after 10 minutes and curve c) is after 20 minutes. Curve d) is after O₂ has been removed by N₂ sparging. These curves show that; 1) the cathodic reduction current is increased and 2) that the anodic reoxidation current disappears. The acceleration of the cathodic current is due to the chemical reaction of O₂ with the anthrahydroquinone (which returns anthraquinone which is re-reduced and hence an accelerated current) while the absence of the reoxidation process indicates that the anthrahydroquinone is consumed in the O₂ reduction reaction. This behavior is identical to that for anthraquinone electrochemistry in protic media in the absence/presence of O₂. FIG. 12d shows the cyclic voltammogram after O₂ has been remover (via N₂ sparging of the solution), it can be seen that the electrochemical behavior returns to its original behavior after removal of O₂.

### Example 3

### Detection of Generated Peroxide

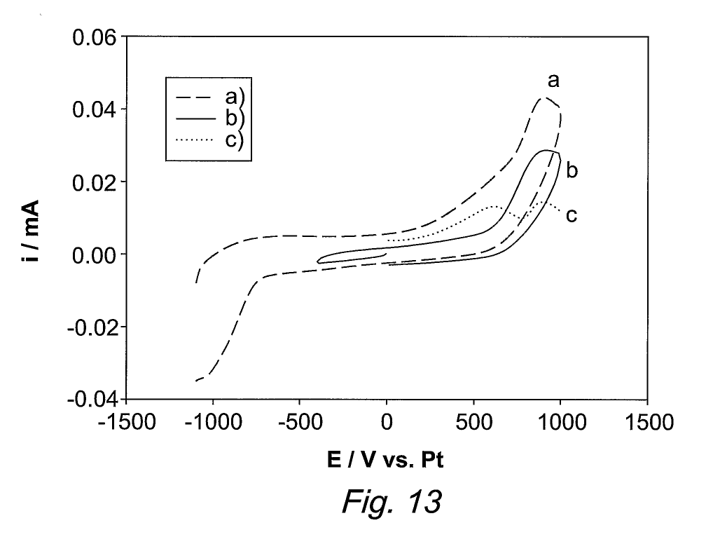

Although the reaction is kinetically slow, peroxide can be oxidised at voltages >0.25 V at carbon electrodes. In this way peroxide generated due to the reaction of O₂ with electrogenerated anthrahydroquinone can be detected. FIG. 13a shows a current-voltage profile for [Bmim⁺] [AQ-COO⁻] in the presence of O₂, while FIG. 13b shows a current-voltage profile also in the presence of O₂ but at less negative voltage limits. In FIG. 13a, the anthrahydroquinone is formed at the negative voltages (cathodic current) whereas in FIG. 13b, anthrahydroquinone is not formed. Comparing FIGS. 13a and 13b, it can be seen that there is an enhanced anodic current in the peroxide oxidation region. Subtracting FIG. 13b from 13a yields FIG. 13c which is the response due to peroxide oxidation (the first peak in FIG. 13c). This demonstrates that peroxide is generated as anthrahydroquinone is generated.

1. A molten salt (Cat⁺An⁻) comprising:
a quinone or quinone derivative as anion or cation, wherein the quinone or quinone derivative has the structure of formula I, II, or III: 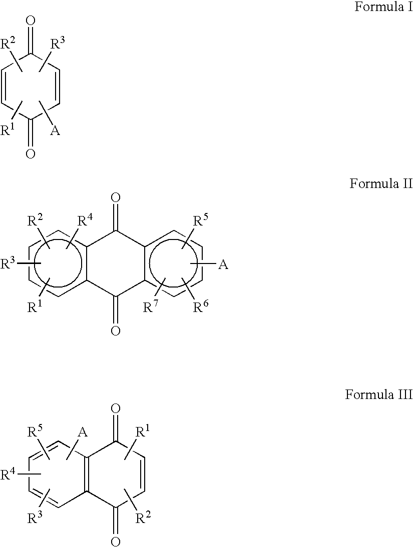
wherein:
R ¹ to R⁷ may independently be A; hydrogen: C₁₋₁₀ linear, branched chain or cyclic alkyl groups; aryl; heterocycles; CN; OH; or NO₂.

2. A molten salt as claimed in claim 1, wherein the quinone or quinone derivative is anionic, and has the structure:

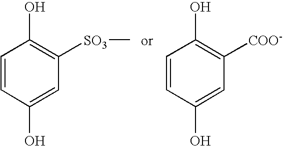

3. (canceled)

4. A molten salt as claimed in claim 1 wherein:
the quinone or quinone derivative is cationic; and
one or more of the ring atoms is a quaternised heteroatom and A represents hydrogen, a C₁₋₁₀ linear, branched chain or cyclic alkyl group, aryl group, a heterocycle group, CN, OH or NO₂.

5. A molten salt as claimed in claim 4 wherein:
each quaternised heteroatom comprises imidazolium, piperidinium, pyridinium, phosphonium, pyrazinium, quaternary amine, ammonium species or derivative thereof and A represents hydrogen; a C₁₋₁₀ linear, branched chain or cyclic alkyl group; an aryl group; a heterocycle group: CN; OH or NO₂.

6. A molten salt as claimed in claim 4 wherein the salt further comprises an anion-selected from the group consisting of PF₆, tetrafluoroborate, bistriflimide, triflate, nitrate, hexafluorophosphate, phosphate, carboxylic acid, thiocyanate and derivatives thereof.

7. A molten salt as claimed in claim 1, wherein the salt comprises N-butyl-N-methyl piperidinium hydroquinone sulfonate, N-octyl-N-methyl piperidinium hydroquinone sulfonate, 1-octyl-4-methyl imidazolium hydroquinone sulfonate, tetradecyltrihexylphosphonium hydroquinone sulfonate, butylmethylpyrrolidinium hydroquinonesulfonate, butylmethylimidazolium hydroquinonesulfonate, or butylmethylimidazolium anthraquinone-2-carboxylate.

8. A process for the production of hydrogen peroxide comprising:
oxidizing a molten salt comprising a hydroquinone or hydroquinone derivative as anion (An⁻) or cation (Cat⁺) to form a corresponding quinone or quinone derivative and produce hydrogen peroxide.

9. The process as claimed in claim 8 further comprising:
reducing a molten salt comprising a quinone or quinone derivative as anion (An⁻) or cation (Cat⁺) to produce the hydroquinone or hydroquinone derivative.

10. The process as claimed in claim 8 wherein the molten salt comprises:
a quinone or quinone derivative as anion or cation, wherein the quinone or quinone derivative has the structure of formula I, II, or III: 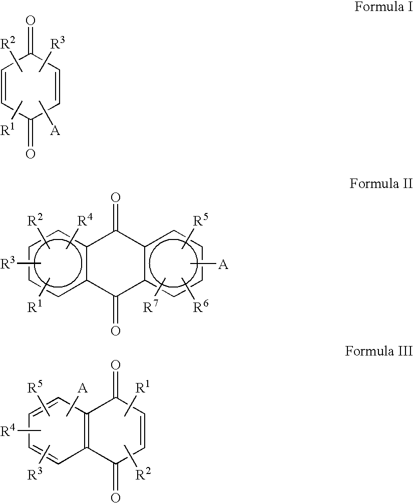
wherein:
R¹ to R⁷ may independently be A; hydrogen; C₁₋₁₀ linear, branched chain or cyclic alkyl groups; aryl; heterocycles; CN; OH; or NO₂.

11. The process as claimed in claim 8 wherein the process is carried out substantially in the absence of any molecular solvent.

12. The process as claimed in claim 9 wherein reducing comprises catalytic hydrogenation or electrolysis.

13. The process as claimed in claim 8 further comprising adding an ionic liquid and/or a solvent comprising one or more of nitriles, alcohols, esters, carbonates, ethers, furans and sulfoxides to the molten salt comprising a hydroquinone or hydroquinone derivative.

14. The process as claimed in claim 13 wherein the ionic liquid comprises imidazolium, pyridinium, piperidinium, phosphorium or quaternary ammonium salts of trilate, bistriflimide, nitrate, hexafluorophosphate or tetrafluoroborate.

15. A method comprising:
using a molten salt in the production of hydrogen peroxide, wherein the salt comprises:
a quinone or quinone derivative as anion or cation, wherein the quinone or quinone derivative has the structure of formula I, II, or III: 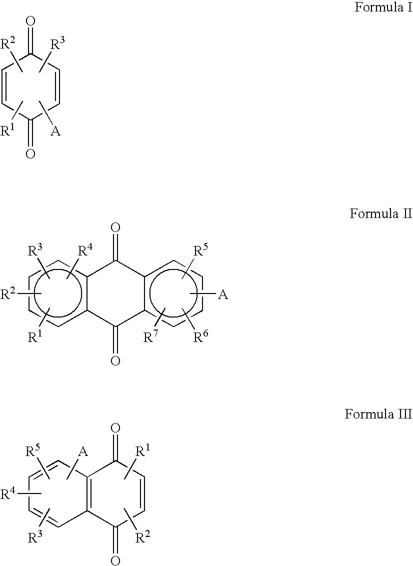
wherein:
R¹ to R⁷ may independently be A; hydrogen; C₁₋₁₀ linear, branched chain or cyclic alkyl groups; aryl; heterocycles; CN; OH; or NO₂.

16. A method of forming a molten salt comprising:
(a) dissolving a first salt nCat⁺Xⁿ⁻, where Cat⁺=a cation, X=Cl⁻, Br⁻ or I⁻ in which case n=1, or X=SO₄²⁻ in which case n=2, in a first organic solvent to form a first solution;
(b) dissolving a second salt bM⁺An^{x-}, where An^{x-}=an anion M=K⁺, Na⁺, Li⁺ or Ag⁺ ad b=1 to 8, in a second organic solvent to form a second solution;
(c) precipitating an inorganic salt (NMXⁿ⁻) by mixing the first and second solutions; and
(d) removing the first and second organic solvents to recover the molten salt (Cat⁺An⁻).

17. The method as claimed in claim 16 wherein one or both of the first and second solvents is selected from the group consisting of acetonitrile, acetone, dimethylformamide, tetrahydrofuran, dimethylsulfoxide and mixtures thereof.

18. A method of preparing a molten salt (Cat⁺An⁻) comprising:
(A) heating, in solid state, a mixture of a carboxylic or sulfonic acid (bH⁺An^{b-}) where b=1 to 8 and a salt nCat⁺Xⁿ⁻ where X=Cl⁻, Br⁻ or I⁻ and n=1; or x=SO₄²⁻ and n=2 to liberate nHXⁿ⁻; and
(B) recovering a molten salt (Cat⁺An⁻.

19. The salt of claim 1, wherein:
the quinone or quinone derivative is anionic; and
A represents SO₃⁻ or COO⁻.

20. The salt of claim 1, wherein:
the quinone or quinone derivative is cationic; and
A comprises represents imidazolium, piperidinium, pyridinium, phosphonium, pyrazinium, quaternary amine, ammonium species or a derivative thereof.

21. The salt of claim 20, wherein:
one or more of R¹-R⁷ comprises imidazolium, piperidinium, pyridinium, phosphonium, pyrazinium, quaternary amine, ammonium species or a derivative thereof.

22. The process of claim 10, wherein:
the quinone or quinone derivative is anionic; and
A represents SO₃⁻ or COO⁻.

23. The process of claim 10, wherein:
the quinone or quinone derivative is cationic; and
A comprises represents imidazolium, piperidinium, pyridinium, phosphonium, pyrazinium, quaternary amine, ammonium species or a derivative thereof.

24. The process of claim 23, wherein:
one or more of R¹-R⁷ comprises imidazolium, piperidinium, pyridinium, phosphonium, pyrazinium, quaternary amine, ammonium species or a derivative thereof.

25. The method of claim 15, wherein:
the quinone or quinone derivative is anionic; and
A represents SO₃⁻ or COO⁻.

26. The method of claim 15, wherein:
the quinone or quinone derivative is cationic, and
A comprises represents imidazolium, piperidinium, pyridinium, phosphonium, pyrazinium, quaternary amine, ammonium species or a derivative thereof.

27. The method of claim 26, wherein:
one or more of R¹-R⁷ comprises imidazolium, piperidinium, pyridinium, phosphonium, pyrazinium, quaternary amine, ammonium species or a derivative thereof.
